(19) United States
(12) Patent Application Publication
(10) Pub. No.: US 2025/0040879 A1
(43) Pub. Date: Feb. 6, 2025

(54) SYSTEM, KIT AND PROCESS FOR DIAGNOSIS AND TREATMENT OF OBSTRUCTIVE SLEEP APNEA USING ORAL APPLIANCE THERAPY

(71) Applicant: 12232821 Canada Inc., Ottawa (CA)

(72) Inventors: Caner KARACEPER, Ottawa (CA); Cem KARACEPER, Ottawa (CA); Prakash CRJ NAIDU, Ottawa (CA); Anida DZAFIC, Ottawa (CA)

(21) Appl. No.: 18/571,059

(22) PCT Filed: Jun. 16, 2022
(86) PCT No.: PCT/CA2022/050964

§ 371 Date: Dec. 15, 2023

Related U.S. Application Data
(60) Provisional application No. 63/211,161, filed on Jun. 16, 2021.

(51) Int. Cl. A61B 5/00

(52) U.S. Cl. CPC A61B 5/4818

(57) ABSTRACT

The present invention provides a system and associated processes and kits for diagnosing and treating mild to moderate obstructive sleep apnea.

### FIELD OF THE INVENTION

The present invention pertains to the field of treatment of sleep disorders and in particular to systems and processes for diagnosis and treatment of obstructive sleep apnea.

### BACKGROUND

Obstructive sleep apnea (OSA) is a potentially serious sleep disorder in which breathing stops involuntarily for brief periods of time during sleep. It causes breathing to repeatedly stop and start during sleep. This type of apnea occurs when your throat muscles intermittently relax and block your airway during sleep. A noticeable sign of obstructive sleep apnea is snoring.

Traditional diagnosis and treatment for obstructive sleep apnea involves testing patients in a clinic/lab setting, followed by having a sleep physician prescribe a suitable treatment. Traditionally, patients are first prescribed continuous positive airway pressure (CPAP) therapy as the first line of treatment for mild to moderate OSA. If CPAP therapy is proven a failure by misuse or ineffectiveness, oral appliance therapy can be proposed as an alternative therapy. Surgery is also a treatment option.

Although research has shown that an oral appliance can be an effective, low risk and low cost treatment for mild to moderate sleep apnea, the process to obtain an oral appliance and undergo test for oral appliance effectiveness can be lengthy and cumbersome, and typically requires physically visiting doctor's offices, dental offices, and/or family doctor offices.

In addition, in comparison to CPAP treatment where the patient and health care professional are able to view real-time sleep data by the use of the CPAP machine and being able to record sleep data which can then be adjusted via cloud by the treating health care professional, prior art oral appliance therapy system have limited capacity for monitoring for oral appliance therapy in real time:

Therefore, there is a need for convenient OSA diagnosis and treatment options using oral appliance therapy that can be monitored in real-time and therefore do not require multiple visits to health care professionals.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a system, kit and process for diagnosis and treatment of obstructive sleep apnea using oral appliance therapy. In accordance with an aspect of the present invention, there is provided a system for diagnosing and treating obstructive sleep apnea (OSA) in a patient, the system comprising: an oral appliance; one or more biosensors configured to measure the one or more sleep parameters; and a sleep application configured to monitor the one or more sleep parameters of the patient during sleep, wherein the application is configured to conduct one or more of a level 1, level 2, level 3 or level 4 sleep study; wherein the system is configured to: measure one or more sleep parameters while the patient is sleeping in a first sleep condition to obtain a first set of sleep parameters; and measure one or more sleep parameters while the patient is sleeping in a second sleep condition to obtain a second set of sleep parameters; compare the first and second sets of sleep parameters to quantify a change in sleep quality/patterns resulting from wearing the oral appliance; and correlate the change in sleep quality/patterns with a diagnosis of OSA in the patient and a determination of the effectiveness of oral appliance therapy for treating the OSA.

In accordance with another aspect of the present invention, there is provided a process for diagnosing and treating obstructive sleep apnea (OSA) in a patient, comprising the steps of: providing an oral appliance to be worn by the patient; conducting a sleep study of the patient while sleeping, wherein the sleep study comprises: measuring one or more sleep parameters while the patient is sleeping in a first sleep condition to obtain a first set of sleep parameters; and measuring one or more sleep parameters while the patient is sleeping in a second sleep condition to obtain a second set of sleep parameters; comparing the first and second sets of sleep parameters to quantify a change in sleep quality/patterns resulting from wearing the oral appliance; and correlating the change in sleep quality/patterns with a diagnosis of OSA in the patient and a determination of the effectiveness of oral appliance therapy (OAT) for treating the OSA.

In accordance with another aspect of the present invention, there is provided a kit for diagnosing obstructive sleep apnea (OSA) in a patient, the kit comprising: equipment for preparing an oral appliance in accordance with the present invention; one or more biosensors configured to measure one or more sleep parameters; a sleep application configured to monitor the one or more sleep parameters of the patient during sleep, wherein the application is configured to conduct one or more of a level 1, level 2, level 3 or level 4 sleep study; and instructions for use.

In accordance with another aspect of the present invention, there is provided a custom oral appliance for use in diagnosing sleep apnea in a subject wearing the appliance, wherein the oral appliance comprises: a lower mandibular portion adapted for fitting engagement with a lower dentition of the patient; an upper maxillary portion adapted for fitting engagement with an upper dentition of the patient; and a connection system configured to connect the upper maxillary portion and the lower mandibular portion to limit the range of movement of the maxillary portion relative to the mandibular portion, wherein the connection system comprises: at least one connecting strap configured for attachment at a first end to the upper maxillary portion and at a second end to the lower mandibular portion, wherein the at least one connecting strap is attached to the upper maxillary portion and the lower mandibular portion via retainer pins located on an outer side of the upper maxillary portion and on an outer side of the lower mandibular portion; wherein the oral appliance is configured for use in conjunction with one or more biosensors configured to measure one or more sleep parameters, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring.

### BRIEF DESCRIPTION OF THE FIGURES

FIG. 1 is schematic diagram of a process for using an oral appliance (OA), biosensors, and sleep application to diagnose obstructive sleep apnea (OSA) and/or determine effectiveness of the OA, in accordance with one embodiment of the invention.

FIG. 2 is schematic diagram of an alternative process for diagnosing obstructive sleep apnea (OSA) and/or determining the effectiveness of an oral appliance carried out by conducting sleep tests without or with an oral appliance (provisional and/or custom), in accordance with one embodiment of the invention.

FIG. 3 is a schematic diagram of a kit comprising the elements used to carry out a process in accordance with an embodiment of the present invention.

FIG. 4 is a schematic diagram of a process for using an oral appliance, including steps for manufacturing the oral appliance, in accordance with an embodiment of the present invention.

FIG. 5 is a schematic diagram of a system of the invention incorporating tele-health aspects, in accordance with an embodiment of the present invention.

FIGS. 6A-6D are schematic diagrams illustrating side and front views of different positional configurations of the mandible (lower jaw) and the maxilla (upper jaw) achievable using an oral appliance (OA), in accordance with an embodiment of the present invention.

FIGS. 7A and 7B are photographs of an oral appliance (OA) suitable for use in and prepared using the processes of the present invention, in accordance with embodiments of the present invention.

FIGS. 8A and 8B are photographs of an oral appliance (OA) suitable for use in and prepared using the processes of the present invention, in accordance with embodiments of the present invention.

### DETAILED DESCRIPTION OF THE INVENTION

The term “obstructive sleep apnea” or “OSA” is used to refer a condition in which breathing stops involuntarily for brief periods of time during sleep (referred to as “apneic episodes”) due to the intermittent relaxation of throat muscles causing blockage of the airway.

The term “positional obstructive sleep apnea” is used to refer to sleep apnea when the majority of apneic episodes can be attributed to sleep position.

The term “oral appliance” is used to refer to a device worn during sleep to support the jaw in a position to help maintain an open upper airway.

The term “provisional appliance” is used to refer to an oral appliance that can be used to provide an assessment of the effect of wearing an oral appliance on OSA in a patient.

The term “mandibular advancement device” is used to refer to an oral appliance that is configured to hold the mandible (lower jaw) in an advanced (pushed forward) position relative to the upper jaw.

The term “positional therapy” is used to refer to a behavioral strategy for treating positional sleep apnea through the application of a stimulus to trigger a patient to maintain a sleep position that reduces or minimizes positional obstructive sleep apnea, or to move from an unfavorable sleep position to a favorable sleep position.

The term “dentition” is used to refer to the patient's teeth and their arrangement in the patient's mouth.

The term “oral appliance therapy” is used to refer to the treatment of OSA using a removable oral appliance in the patient's mouth during sleep to prevent the airway from collapsing by either holding the tongue or supporting the jaw in a forward position.

The terms “patient” and “user” are used interchangeably in the present disclosure, and refer to the person who is being diagnosed and/or treated for OSA.

The term “health care professional” may be used to refer generally to a professional who has experience with administering and supervising treatment for and management of sleep disorders and may include a sleep physician, a dental professional, or a general practitioner.

Unless defined otherwise, all technical and scientific terms used herein have the same meaning as commonly understood by one of ordinary skill in the art to which this invention belongs.

The present invention provides systems and associated processes and kits for the diagnosis and treatment of obstructive sleep apnea (OSA) in a patient determined to be suffering from this condition. The systems are designed to provide a convenient, inexpensive and relatively expedient/quick means to arrive at an initial diagnosis of OSA and a determination of the effectiveness of oral appliance therapy for treating the OSA, as well as convenient access to a suitable treatment regimen over the subsequent stages.

In accordance with the present invention, the system is configured to facilitate a determination, or diagnosis, of whether the patient suffers from OSA. In one embodiment, the system is also configured to facilitate a determination of whether the patient is eligible for treatment of the OSA using an oral appliance, also referred to as oral appliance therapy (OAT).

In one embodiment, the system is configured to treat the OSA using positional therapy (PT) using stimulus provided by the oral appliance. In this embodiment, the positional therapy is referred to as positional oral appliance therapy (POAT).

In accordance with the present invention, the system employs the use of a provisional oral appliance in conjunction with a sleep application to provide a first determination of whether the patient suffers from OSA. In a preferred embodiment, the OSA is mild to moderate OSA.

In a preferred embodiment, the sleep application is configured to conduct one or more of a level 1, level 2, level 3 and level 4 sleep study. A level 1 sleep study is carried out in a facility such as a sleep lab, under supervision by a qualified sleep technologist, wherein sleep parameters such as brain activity, heart rate, breathing activity, blood oxygen levels, snoring, eye and limb movements are monitored. A level 2 sleep study is used to monitor the same sleep parameters as a level 1 study, but it is carried out at home. A level 3 sleep study is also carried out at home, and monitors blood oxygen levels, heart rate, breathing activity, and snoring; brain and muscle activity are not monitored. A level 4 sleep study is carried out by monitoring blood oxygen and heart rate to screen for sleep apnea.

Accordingly, in a preferred embodiment, the system of the present invention includes a provisional oral appliance, as well as a custom oral appliance upon determination that the patient is a candidate for OAT.

In another preferred embodiment, the system of the present invention also includes a sleep application configured to monitor one or more sleep parameters of the patient during sleep, while the patient is wearing an oral appliance, wherein the oral appliance may be a provisional oral appliance or a custom oral appliance. In a preferred embodiment, the sleep parameters are monitored in real-time. In a preferred embodiment, the sleep application is configured to conduct one or more of a level 1, level 2, level 3 and level 4 sleep study.

In a preferred embodiment, the oral appliance is adjustable to provide different position of the bottom component relative to the upper component of the appliance. For example, the oral appliance may be adjustable to move the patient's lower jaw in one or more of a forward, rearward, lateral, upward, and downward direction relative to the upper jaw.

In order to determine whether the patient suffers from OSA, a sleep test is carried out by monitoring a patient while sleeping in a first sleep condition to obtain a first set of sleep parameters, and again while the patient is sleeping in a second sleep condition to obtain a second set of sleep parameters. In accordance with the present invention, the sleep test may be any of a level 1, level 2, level 3 or level 4 sleep study, or a combination thereof.

In one embodiment, the first sleep condition comprises sleeping without a provisional oral appliance and the second sleep condition comprises sleeping with a provisional oral appliance.

In another embodiment, the first sleep condition comprises sleeping with a provisional oral appliance in a first state of adjustment, and the second sleep condition comprises sleeping with a provisional oral appliance in a second state of adjustment.

In one embodiment, the first state of adjustment is a non-protrusive state where the upper jaw and lower jaw are in their natural relaxed relative positions, and the second state of adjustment is a protrusive state where the upper jaw and lower jaw are in positions displaced from their natural relative positions. In one embodiment, the unnatural relative position is determined according to the requirements of the patient.

The two sets of sleep parameters are then compared to determine the effect of sleeping under the second sleep condition and whether the effect is beneficial, i.e., an improvement over sleeping under the first sleep condition.

This comparison can therefore be used to facilitate a determination of whether the subject is a candidate for treatment using oral appliance therapy, if there is a measurable change in sleep quality upon wearing of the oral appliance.

In a preferred embodiment, the sleep test is carried out using the sleep application to actively monitor one or more sleep parameters while the patient is sleeping with the oral appliance in a first sleep condition to obtain a first set of sleep parameters, and again while the patient is sleeping with the oral appliance in a second sleep condition to obtain a second set of sleep parameters. The system then compares the first and second sets of sleep parameters to quantify a change in sleep quality/patterns resulting from wearing the oral appliance, and correlates the change in sleep quality/patterns with a diagnosis of OSA in the patient and/or a determination of the effectiveness of OAT for treating the OSA. In one embodiment, the oral appliance is a provisional oral appliance.

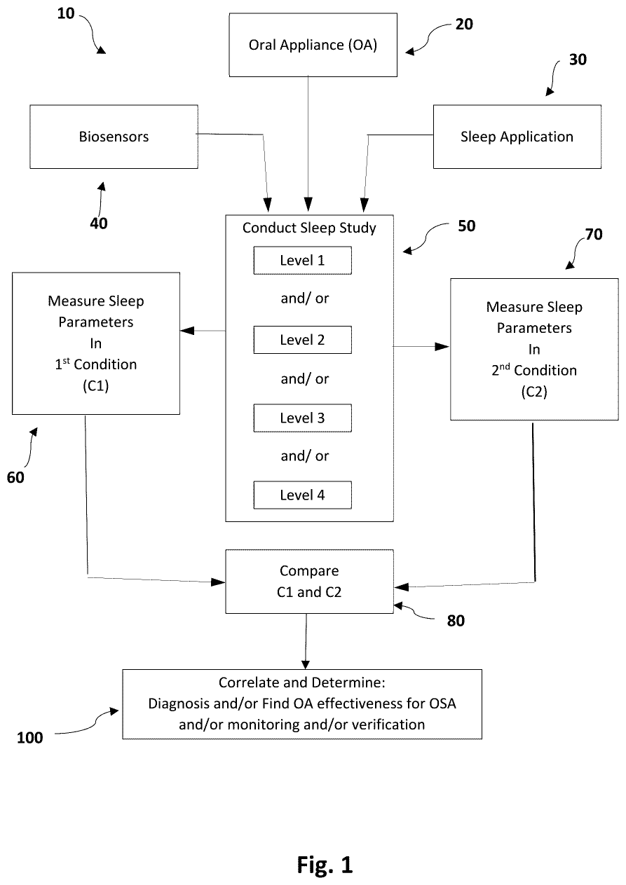

FIG. 1 is a schematic diagram of an embodiment of a process 10 of the invention employing oral appliance (OA) 20, biosensors 40, and sleep application 30 to conduct a sleep study on a patient wherein sleep parameters are measures while the patient is sleeping with the oral appliance under a first sleep condition C1 and again while under a second sleep condition C2. The two sets of sleep parameters C1 and C2 are compared 80 and if a change in the parameters is observed, the difference is used to determine 100 provide a diagnosis of OSA and/or the effectiveness of OAT for treating the OSA.

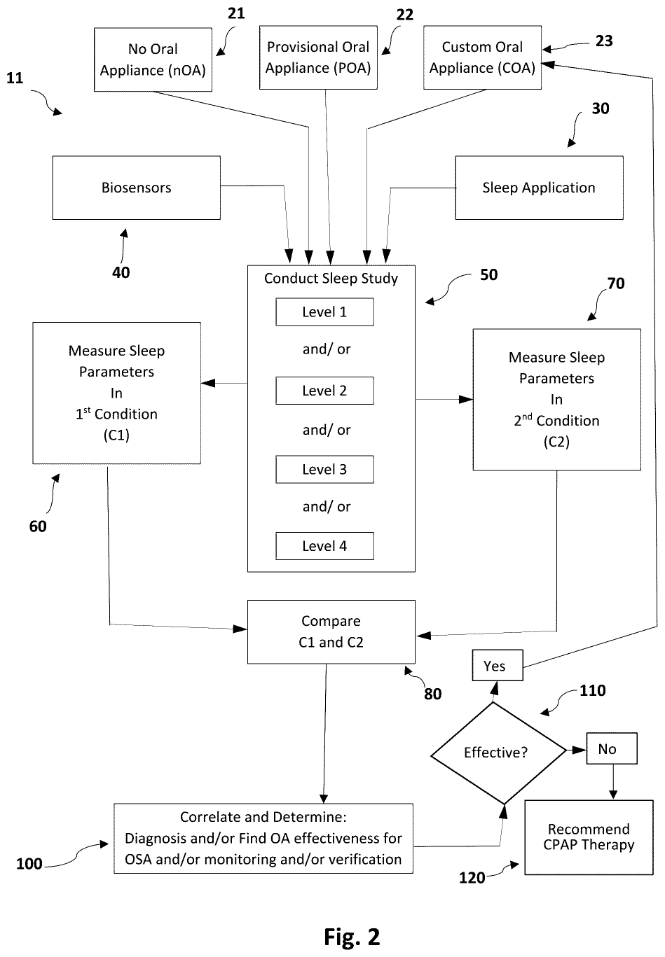

FIG. 2 is a schematic diagram of a further embodiment of a process 11 of the invention in which the process is carried out while the patient is first wearing no oral appliance (nOA) 21, then while wearing a provisional oral appliance (POA) 22, then again while wearing a custom oral appliance (COA) 23. Under each of these conditions, the process is carried out in conjunction with biosensors 40 and sleep application 30 to conduct one or more sleep studies 50 on the patient. In one embodiment, the first sleep condition 60 may involve wearing no oral appliance (nOA) 21 and the second sleep condition 70 may involve wearing the POA 22. The first and second sleep conditions C1 and C2 are compared 80 and an observed difference may be used to determine 100 whether the POA 22 is effective in treating OSA. If it is found to be effective 110, this information can be used to recommend the manufacture of a custom oral appliance 23. If the use of POA 22 is observed to not be effective in treating OSA, a recommendation 120 for continuous positive airway pressure (CPAP) therapy may be made.

In one embodiment, the comparison of the first and second sets of sleep parameters is carried out by one or more health care professionals, such as a sleep physician, a dental professional, a general practitioner. In one embodiment, the health care professional carries out the comparison by remotely accessing the sleep parameter data via the sleep application. In one embodiment, the comparison is carried out by the patient themselves. In one embodiment, the comparison is carried out by the sleep application.

Although the present disclosure describes the monitoring of sleep parameters during first and second sleep conditions, it is within the scope of the present invention that the system is configured to monitor the patient while sleeping under multiple different sleep conditions, such as the different conditions achieved during a process of adjusting an oral appliance between different positional states to obtain the optimum configuration for treatment of OSA using the oral appliance.

It is also within the scope of the present invention that monitoring of the first and subsequent sleep conditions can be carried out over multiple sleep sessions, either consecutively or intermittently.

For example, in one embodiment, once it has been determined that the subject does suffer from OSA, the oral appliance can be adjusted to different positional states over the course of multiple sessions, to determine the relative effect, either beneficial or detrimental, observed for each respective positional state.

In a preferred embodiment, the sleep application is configured to interface with a wearable or non-wearable smart device configured to measure the one or more sleep parameters using one or more biosensors.

In preferred embodiment, the sleep application is provided as a mobile application executable on a smart device such as personal computer, a tablet, a mobile phone or a smart watch.

In one embodiment, the system includes one or more biosensors configured to measure the one or more sleep parameters. In one embodiment, the sleep parameters are selected from one or more of heart rate, breathing rate, head movement, body movement, bite force, actigraphy, oximetry, apnea hypopnea index (AHI), peripheral arterial tone (PAT), cardiac data, chest expansion, respiratory air flow, sleeping position, body temperature, and snoring. In one embodiment, the biosensors are associated with a wearable device such as a smart watch, a chest strap, a head strap, a fingertip monitor and/or the provisional or custom oral appliance. In one embodiment, the biosensors are associated with a non-wearable device that can be located in proximity to the sleeping patient, for example, under the pillow or next to or under the bed. In one embodiment, the biosensors are located in or on the provisional or custom oral appliance.

In one embodiment, where the system is employed to treat the OSA using positional oral appliance therapy (POAT), the sleep position of the patient is monitored using the biosensors in the system, and when an unfavorable sleep position is identified, a stimulus is applied to the patient to urge them to assume a favorable sleep position. In one embodiment, the stimulus is a vibration provided by a wearable device or by the oral appliance.

In order to obtain a diagnosis of OSA and a determination of eligibility for subsequent treatment using oral appliance therapy (OAT), the patient must be provided with a provisional oral appliance that is properly fitted to the patient's dentition to be worn during sleep. In one embodiment, the provisional oral appliance is self-fitted by the patient, or fitted by a qualified health care professional.

In one embodiment, the provisional oral appliances are prepared by taking an impression of the patient's dentition, and using the resulting impression to form a solid mold of the dentition which is then used to fabricate the provisional oral appliance.

In one embodiment, the provisional oral appliance is a moldable oral appliance. In one embodiment, the oral appliance is manufactured from a heat moldable material, wherein the material is preheated to a temperature sufficient to soften the moldable material to receive an impression of the user's teeth, and return to a hardened state upon cooling.

In one embodiment, the provisional or custom oral appliance is configured to fit within the patient's mouth while holding the patient's jaw in a position suitable for determining whether oral appliance therapy is a treatment option. In one embodiment, the jaw position is determined by a health care professional. In one embodiment, the jaw position is determined by the patient.

In one embodiment, the provisional or custom oral appliance is adjustable to vary the degree of one or more of a forward, rearward, lateral, upward and downward thrust of the patient's lower jaw.

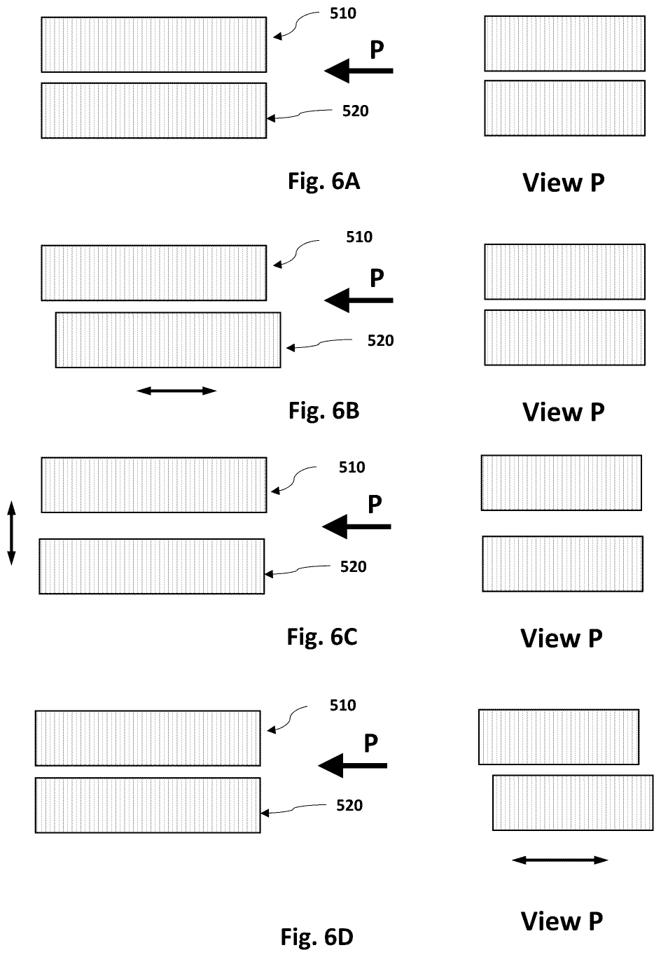

FIGS. 6A to 6D are schematic diagrams illustrating side elevation views and corresponding front views P of a two-part oral appliance (OA), one part of which is configured to fit to mandible (lower jaw) 520 and the other part of which is configured to fit to the maxilla (upper jaw) 510.

FIG. 6A depicts the oral appliance holding the upper jaw 510 and lower jaw 520 in a neutral positional configuration. FIG. 6B depicts the oral appliance holding the lower jaw 520 in an advanced (pushed forward) position relative to the upper jaw 510. FIG. 6C depicts the oral appliance holding the lower jaw 520 in an open positional configuration, increasing the gap relative to the upper jaw 510. FIG. 6D depicts the oral appliance holding the lower jaw 520 in a laterally displaced configuration relative to the upper jaw 510.

In one embodiment, the provisional or custom oral appliance can be used to determine the patient's centric bite, edge to edge bite, and also how far the patient can push their lower jaw forward, rearward, upward, downward, or to each side. These measurements can be recorded via a tele-health platform available through the sleep application for reference in preparing a custom oral appliance. Once the optimal relative position for treatment of OSA has been determined using the provisional oral appliance, this information is retained and applied for the fabrication of the custom oral appliance.

In addition to providing a diagnosis of OSA, the comparison of the first and second (or more) sets of sleep parameters can also provide an indication of the effectiveness of an oral appliance for mitigating OSA in the patient, to help determine whether the patient is a possible candidate for oral appliance therapy.

Accordingly, in a preferred embodiment of the present invention, the provisional oral appliance is also employed to determine whether the patient is eligible for treatment using oral appliance therapy.

In one embodiment, an oral appliance therapy regimen may be carried out using the provisional oral appliance. In such an embodiment, it may be preferable for the provisional oral appliance to be a smart provisional oral appliance, i.e., an appliance that has been fitted with one or more biosensors that monitor and transmit data in real-time.

Alternatively, once a determination has been made that the patient is a candidate for oral appliance therapy, the patient may be advised to obtain a custom oral appliance for treatment over the longer term. In this embodiment, the oral appliance therapy would be carried out using a custom oral appliance fitted with one or more biosensors.

Accordingly, in one embodiment, the system is provided with a kit containing the equipment required to facilitate preparation of a custom oral appliance.

In one embodiment, the equipment includes an impression kit to take an impression of the patient's dentition, which can then be used to prepare the custom oral appliance. In such an embodiment, the equipment required to prepare a custom oral appliance comprises a molding material with which an impression of the patient's dentition can be obtained.

In one embodiment, the resulting impression can be used directly to prepare a cast of the patient's dentition, which can then in turn be used to prepare the custom oral appliance.

In one embodiment, the impression of the patient's dentition is taken using any molding material as in known in the art, and using the resulting impression to form a solid cast of the dentition which is then used to fabricate the provisional oral appliance.

In one embodiment, the molding material is a heat activated moldable material, wherein the material is heated to initiate a curing process, and which reaches its final hardened state upon curing. In one embodiment, the molding material is a chemically or catalytically activated moldable material, wherein the curing process is initiated by the addition of a catalyst to a moldable base material, and which reaches its final hardened state upon curing. In one embodiment, the moldable material changes color or appearance to indicate when it has reached a final cured state.

In one embodiment, the custom oral appliance is prepared using the provisional appliance as a mold.

In one embodiment, the impression of the patient's dentition is scanned with a 3-D scanner to obtain a digital image of the patient's dentition, which is then used to prepare the custom oral appliance.

In one embodiment, the 3-D scan of the impression is obtained using a CT scanner or a cone beam CT (CBCT) scanner.

In one embodiment, the equipment required to prepare a custom oral appliance is an intra-oral 3-D scanner, configured to scan the patient's mouth directly to obtain a digital 3-D image of the patient's dentition, which is then used to prepare the custom oral appliance.

In one embodiment, the 3-D intra-oral image is obtained using an imaging device in communication with a device such as a smart phone, tablet or personal computer. In one embodiment, the scanning process is managed using a mobile application on the device. In one embodiment, the communication between scanner and device is carried out on a wireless or Bluetooth connection. In one embodiment, the communication between scanner and device is carried out over a wired connection. In one embodiment, the imaging device is an optical camera. In one embodiment, the imaging device is a laser scanner.

In one embodiment, the 3-D image of the patient's dentition is provided as an STL file or in any suitable 3D file format.

Once a 3-D image of the dentition has been obtained, the custom oral appliance can be prepared using any method for forming three dimensional objects as are known in the art, including but not limited to, 3-D resin and powder printing methods, thermoforming methods, and milling methods.

In one embodiment, the custom oral appliance may be manufactured using any CAD/CAM technology as is known in the art.

In one embodiment, the biosensors are incorporated after formation of the custom oral appliance. In one embodiment, the biosensors are incorporated before final formation of the custom oral appliance.

In one embodiment, if the patient has been diagnosed with OSA and a determination has been made regarding whether the OSA is treatable using OAT, the patient is directed to contact a dental professional for the manufacture of a custom oral appliance.

In accordance with one embodiment, the system further includes a tele-health support platform accessible via a mobile application interface. The tele-health support platform allows the patient to be monitored remotely and in real time by a network of health care professionals, thus providing access to ongoing assessments of the effectiveness of the oral appliance therapy in a timely manner and without requiring the inconvenience and associated cost of multiple visits to the health care professional's medical offices or a sleep lab.

In such an embodiment, the oral appliance, either provisional or custom, is preferably a smart oral appliance, embedded with one or more biosensors configured to measure sleep parameters. The system comprising the smart oral appliance, together with the sleep application, and optionally in conjunction with wearable or non wearable technology, is configured to measure sleep parameter data in real time.

Accordingly, in one embodiment, there is provided a custom oral appliance for use in diagnosing sleep apnea in a subject wearing the appliance, wherein the oral appliance comprises one or more biosensors configured to measure one or more sleep parameters, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring. In one embodiment, the custom oral appliance also comprises an acoustic recorder to record audible sounds during sleep, such as snoring or talking.

The sleep parameter data can be transmitted and accessed in real time by a health care professional via the tele-health support platform, or it can be stored for later reference and analysis.

In one embodiment, sleep parameter data obtained from biosensors in the oral appliance are transmitted in real time via the tele-health support platform to be monitored by a health care professional, who can issue instructions to adjust the oral appliance via the tele-health support platform based on the sleep parameter data thus obtained. In one embodiment, the instructions are issued to the oral appliance for automatic adjustment. In one embodiment, the instructions are issued to the patient to make the adjustments to the oral appliance

In one embodiment, the system is further configured to facilitate a positional sleep therapy program to treat sleep apnea. In this embodiment, the system comprises a means for alerting the subject to change sleep position when the system has detected that the subject is sleeping in an unfavorable position, thus encouraging the subject into a favorable sleep position. In one embodiment, the alerting means provides a gentle vibration when the subject has been sleeping on their back to encourage them to move onto their side.

In one embodiment, the alerting means is located in any of the wearable devices such as a smart watch, a chest strap, a head strap, a fingertip monitor, or in the oral appliance itself. In one embodiment, the alert is triggered via the tele-health platform.

In addition to providing a means for monitoring and accessing sleep parameter data, the tele-health platform can also be used to manage the process of manufacturing the custom oral appliance. For example, those embodiments in which the system comprises a 3-D oral scanner, the patient can obtain the 3-D scan of their dentition, and upload the image to the platform, where it can be accessed by the dental professional responsible for manufacturing the custom oral appliance.

The tele-health support platform also allows for remote communication between the patient and the health care professionals, thus facilitating all stages of treatment, diagnosis and continued care programs.

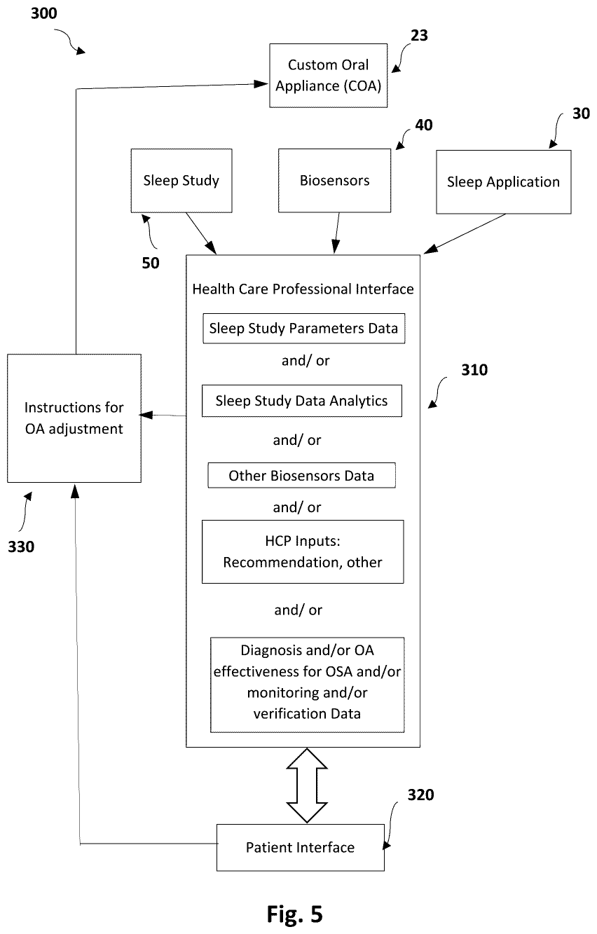

FIG. 5 is a schematic diagram of one embodiment of a tele-health system 300, including health care professional (HCP) interface 310 in interaction with patient interface 320. Using the HCP interface 310, the health care professional is able to access informational inputs provided by sleep study 50, biosensors 40, and sleep application 30. These informational inputs may therefore include one or more of sleep study parameter data, sleep study data analytics, and biosensor data. Using the informational inputs provided by the HCP interface, the health care professional is able to make a diagnosis of OSA and/or determine the effectiveness of the oral appliance for treating OSA. The HCP interface also allows the health care professional to provide inputs to the patient regarding, for example, instructions regarding adjustments 330 to the custom oral appliance 23, either by the health care professional, by the patient, or both.

Although not shown in FIG. 5, the tele-health system 300, in one embodiment, employs Wi-Fi, Bluetooth or other suitable wireless communication means to facilitate the transfer of information from the sleep study 50, biosensors 40 and sleep application 30 to the HCP interface 310, and between the HCP interface 310 and patient interface 320. In another embodiment, the tele-health system 300 is managed through a cloud platform. In one embodiment, the instructions for OA adjustment are transmitted and executed in real-time.

In accordance with a further aspect of the invention, there is provided a kit containing the components required for diagnosing obstructive sleep apnea (OSA) in a patient in the home setting. It is, however, within the scope of the present invention to carry out one or more the steps of the process in a clinical or lab setting.

Accordingly, in one embodiment, the kit comprises the equipment required to prepare an oral appliance suitable for use in diagnosing OSA in a patient.

In accordance with the present invention, the kit further comprises a sleep application configured to monitor the one or more sleep parameters of the patient during sleep. In a preferred embodiment, the sleep application is a mobile application configured to interface with a wearable smart device configured to measure the one or more sleep parameters using one or more biosensors. In accordance with the present invention, the sleep test may be any of a level 1, level 2, level 3 or level 4 sleep study.

In one embodiment, the kit also comprises a wearable smart device selected from a smart watch, a chest strap, a head strap, and a fingertip monitor, and/or a non-wearable smart device that can be located in proximity to the sleeping patient.

In accordance with the present invention, the kit further comprises an impression kit for preparation of a mold of the patient's teeth, wherein the mold is used to manufacture a custom oral appliance.

In one embodiment, the impression kit comprises a molding material that can be preheated to a temperature sufficient to soften the moldable material to receive an impression of the patient's dentition, and return to a hardened state upon cooling. In one embodiment, the impression of the patient's dentition is taken using any molding material as in known in the art, and the resulting impression is used to form a solid cast of the dentition which is then used to fabricate the oral appliance.

In one embodiment, the impression kit comprises a molding material that is a heat activated moldable material, wherein the material is heated to initiate a curing process, and which reaches its final hardened state upon curing. In one embodiment, the molding material is a chemically or catalytically activated moldable material, wherein the curing process is initiated by the addition of a catalyst to a moldable base material, and which reaches its final hardened state upon curing. In one embodiment, the heat activated moldable material changes color or appearance to indicate when it has reached a final cured state.

In one embodiment, the kit also comprises a 3-D oral scanner for scanning the interior of a patient's mouth to obtain a 3-D image for the preparation of a custom oral appliance. In one embodiment, the 3-D image of the patient's dentition is provided as an STL file or any suitable 3D file format.

In one embodiment, the kit comprises an imaging device in communication with a device such as a smart phone, tablet or personal computer for obtaining the 3-D intra-oral image. In one embodiment, the scanning process is managed using a mobile application on the device. In one embodiment, the communication between scanner and device is carried out on a wireless or Bluetooth connection. In one embodiment, the communication between scanner and device is carried out over a wired connection. In one embodiment, the imaging device is an optical camera. In one embodiment, the imaging device is a laser scanner.

In one embodiment, the kit also comprises one or more of a sleep mask, ear plugs, cleaning kit, a bite-repositioner, and/or chewing gum. A bite-repositioner is a small device or appliance to bring the patient's jaw back into its natural bite setting after wearing the oral appliance for an extended period of time.

In another embodiment, the kit comprises a provisional oral appliance of a suitable size according to the requirements of the patient. By providing a range of kits, each having an oral appliance in one of a range of sizes, it can be ensured that an appliance of suitable size can be selected and used for the subsequent diagnostic and treatment processes.

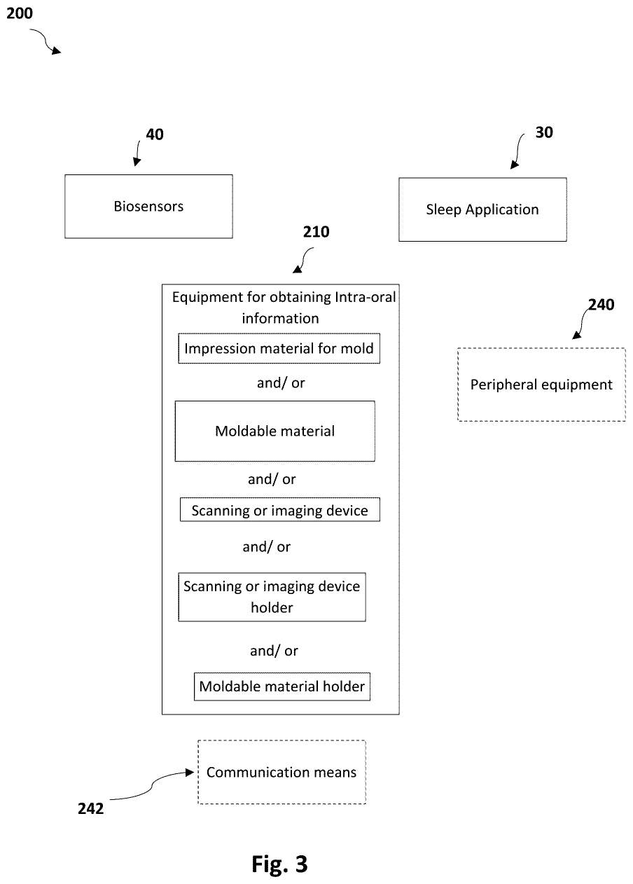

FIG. 3 is a schematic diagram to illustrate a kit 200 according to one embodiment of the invention. In this embodiment, the kit comprises the equipment 210 required to manufacture an oral appliance, including one or more of: impression materials to form a mold of the patient's dentition, moldable material that is thermo, chemically or catalytically setting, means to hold the moldable materials in the oral cavity of a patient to enable obtaining of molded impressions, a scanning or imaging device, and means to hold the device in the oral cavity of a patient to enable obtaining of scans and/or images. Also included in kit 200 are biosensors 40 and sleep application 30. Optional peripheral equipment 240 useful to facilitate the process and for the comfort of the patient may also be included, for example, one or more of a sleep mask, ear plugs, cleaning kit, bite repositioner, jaw position measuring tool, and chewing gum, as well as communication means 242. The communication means may facilitate transmission of information between one component of the kit and another component, for example, between scanner and mobile application as stated above. The communication means may also facilitate unidirectional and/or bi-directional transmission of information between a user patient and a health service provider, for example, bio-sensor information for diagnostics.

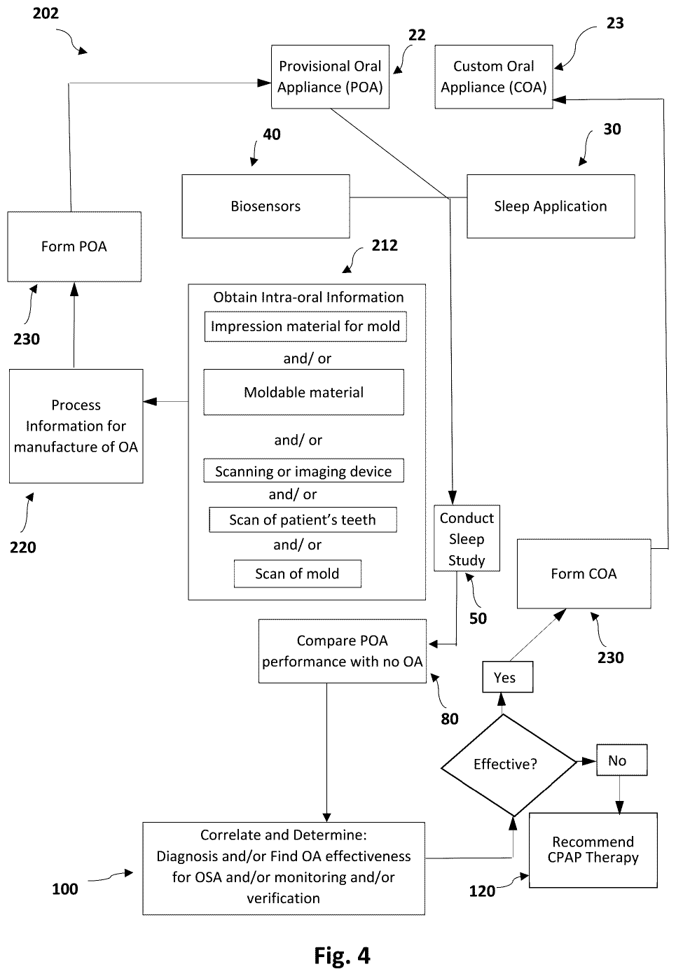

FIG. 4 is a schematic diagram of a process 202 for manufacturing and using an oral appliance, including steps for manufacturing the oral appliance, in accordance with an embodiment of the present invention FIG. 4 employs kit 200 as illustrated in FIG. 3.

As shown in this embodiment, intra-oral information is obtained 212 using equipment for obtaining intra-oral information and processed 220 to provide the parameters used to form 230 provisional oral appliance (POA) 22. POA 22 may be formed by any suitable method such as herein described.

Once POA 22 has been manufactured, sleep study 50 may be conducted using biosensors 40 and sleep application 30. Based on a comparison 80 of sleep study data obtained without an oral appliance (nOA) and with a provisional oral appliance (POA), a correlation and determination 100 may be made to ascertain the effectiveness of Oral Appliance Therapy (OAT). If it is determined that the OAT is found to be effective, the patient may be recommended to continue with OAT, with treatment using a custom oral appliance (COA) 23. The process uses the information obtained in 220 to enables manufacture of a COA 230. If OAT is determined not to be effective in treating OSA, a recommendation 120 is made to switch over to Continuous Positive Airway Pressure (CPAP) Therapy.

In accordance with a further aspect of the invention, there is provided a process for diagnosing [and treating] obstructive sleep apnea (OSA) in a patient. In a preferred embodiment, the process comprises the steps of providing an oral appliance to be worn by the patient, followed by conducting a sleep study of the patient while sleeping, wherein the sleep study comprises measuring one or more sleep parameters while the patient is sleeping in a first sleep condition to obtain a first set of sleep parameters, and measuring one or more sleep parameters while the patient is sleeping in a second sleep condition to obtain a second set of sleep parameters. The first and second sets of sleep parameters are then compared to quantify a change in sleep quality/patterns resulting from wearing the oral appliance, and the change in sleep quality/patterns is correlated with a diagnosis of OSA in the patient and/or a determination of the effectiveness of OAT for treating the OSA. In accordance with the present invention, the sleep test may be any of a level 1, level 2, level 3 or level 4 sleep study.

In one embodiment, the first sleep condition comprises sleeping without the oral appliance and the second sleep condition comprises sleeping with the oral appliance. In one embodiment, the oral appliance is a provisional oral appliance.

In another embodiment, the first sleep condition comprises sleeping with the oral appliance in a first state of adjustment, and the second sleep condition comprises sleeping with the oral appliance in a second state of adjustment. In this embodiment, the oral appliance can be a provisional or custom oral appliance.

In one embodiment, the first state of adjustment is a non-protrusive state where the upper jaw and lower jaw are in their natural relaxed relative positions, and the second state of adjustment is a protrusive state where the upper jaw and lower jaw are in positions displaced from their natural relative positions. In this embodiment, the oral appliance can be a provisional or custom appliance.

The two sets of sleep parameters are then compared to determine the effect of sleeping under the second sleep condition and whether the effect is beneficial, i.e., an improvement over sleeping under the first sleep condition.

If the change in sleep quality/patterns indicates an improvement in sleep quality, a diagnosis of OSA that is treatable using an oral appliance is indicated. In one embodiment, the change in sleep quality/patterns may be further correlated with a determination of the effectiveness of a provisional oral appliance for treatment of the OSA using OAT.

In one embodiment, the change in sleep quality/patterns may be further correlated with a prediction of the effectiveness of a custom oral appliance for treatment of the OSA using OAT.

In one embodiment, wherein upon a positive diagnosis of OSA in the patient and a determination of the effectiveness of OAT for treating the OSA, the process further comprises providing an impression kit to make a mold of the patient's teeth, and using the mold to manufacture a custom oral appliance. In an alternative embodiment, the process comprises the step of using an impression of the provisional oral appliance to prepare the custom oral appliance.

In one embodiment, wherein upon a positive diagnosis of OSA in the patient and a determination of the effectiveness of OAT for treating the OSA, the process further comprises obtaining a 3-D digital scan of the patient's teeth, and using the 3-D digital scan to manufacture a custom oral appliance.

In one embodiment, the 3-D scan is obtained by a health care professional. In one embodiment, the 3-D scan is obtained by the patient. In one embodiment, the 3-D scan is obtained using an optical imaging device in communication with a device such as a smart phone, tablet or personal computer. In one embodiment, the scanning process is managed using a mobile application on the device. In one embodiment, the communication between scanner and device is carried out on a wireless or Bluetooth connection. In one embodiment, the communication between scanner and device is carried out over a wired connection

In one embodiment, the process further comprises providing a tele-health platform configured to provide an interface with one or more health care professionals, whereby the health care professional can monitor the one or more sleep parameters using the tele-health platform in real time.

In one embodiment, based on sleep parameter data thus obtained, the health care professional(s) can issue instructions to adjust the oral appliance via the tele-health support platform. In one embodiment, the instructions are issued to the oral appliance for automatic adjustment. In one embodiment, the instructions are issued to the patient to make the adjustments to the oral appliance

In one embodiment, the necessary oral appliance adjustments are determined using an artificial intelligence approach based on the sleep parameter data.

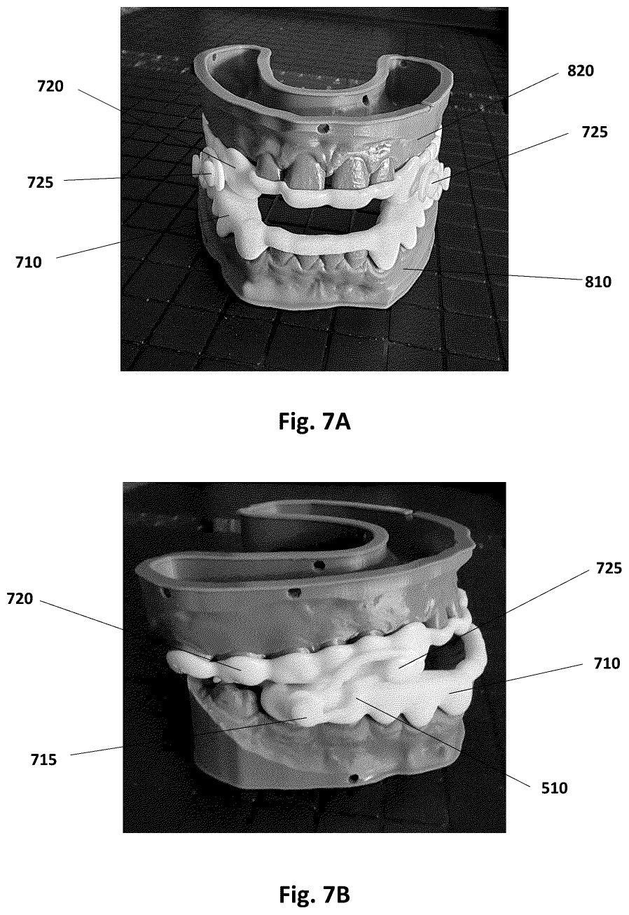

FIGS. 7A-B and 8A-B are photographs of an oral appliance (OA) suitable for use in and prepared using the processes of the present invention. In this embodiment, the oral appliance comprises lower mandibular portion 710 adapted for fitting engagement with the patient's lower dentition 810 and upper maxillary portion 720 adapted for fitting engagement with the upper dentition 820, as illustrated in FIG. 7A (front view) and FIG. 7B (side view).

The oral appliance also comprises a connection system configured to connect upper maxillary portion 720 and lower mandibular portion 710 to limit the range of movement of the maxillary portion relative to the mandibular portion. The connection system comprises connecting straps 750 attached at one end to upper maxillary portion 720 and at the other end to lower mandibular portion 710. In a preferred embodiment, the oral appliance includes two connecting straps 750.

In one embodiment, connecting straps 750 are attached to the upper and lower portions via retainer pins 715, 725 located on the outer sides of respective mandibular and maxillary portions 710, 720. In a further embodiment, the retainer pins are formed integrally as part of the respective mandibular or maxillary portions.

The retainer pins may be structurally integrated with the respective mandibular or maxillary portions during manufacturing by molding, milling, 3D printing or additive manufacturing or other fabrication methods. Alternatively, other approaches also may be deployed such as affixing the pins into the mandibular or maxillary portions by adhesion, snap fit into receptor holes, or by screwing into threaded receptor holes by having matching threads in the pins, or a combination of such approaches. FIG. 7B illustrates a view of a strap assembled with the mandibular or maxillary portions using retainer pins 715, 725.

FIG. 7B illustrates a view of the oral appliance assembly wherein the strap is retained on the maxillary portion of the oral appliance and mandibular portion of the oral appliance using a pair of retainer pins that have pin heads that are larger in diameter than the stems of the retainer pin and the slot in the strap through which the retainer pin slides. In one embodiment, the pin heads are an integral part of the pins. In an alternative embodiment, the pin heads are attached to the pin stems by means such as threads, snap fit, and other methods. The shapes of the retainer pins and the pin heads are shown as circular cylindrical in the embodiment illustrated in the FIGS. 7A-B and 8A-B, however, those skilled in the art may appreciate that several other conceivable shapes are possible, and all such shapes are included within the scope of the present invention.

In one embodiment, the system is provided with a set of straps of differing lengths to enable customization of an oral appliance having the degree of mandibular advancement that is determined to work most effectively for a particular user. Typically, the relative positions of the pins located on the mandibular and maxillary portions of the oral appliance are such that use of shortest strap results in maximum mandibular advancement, whereas the use of longest strap results in minimum mandibular advancement. Different strap lengths may be tried in sequence to determine the optimal degree of mandibular advancement for a particular user, taking into consideration factors such as clearance of airway passage, and comfort of use including reduction or management of side effects such as temporomandibular joint (TMJ) pain.

In one embodiment, the configuration of the strap may have a relatively thicker boss area surrounding the pin hole to provide better strength and thus resistance to tear due to the mandibular advancement force reported in the art as approximately 1 to 1.2 Newtons per millimeter of mandibular advancement (for example, 1.18 Newtons per millimeters as per J Cohen-Levy, B Petelle, J Pinguet, E Limerat, B Fleury, Sleep Breath, 2013 May; 17 (2): 781-9. doi: 10.1007/s11325-012-0765-4. Epub 2012 Sep. 11).

In one embodiment, the user may be provided with a kit comprising 2 to 10 straps, each of a different length. Optionally, the straps may be provided in different colours to indicate the degree of mandibular advancement, for example, red straps may indicate maximum mandibular advancement, yellow straps may indicate medium mandibular advancement, and green straps may indicate minimum mandibular advancement. The colour coding makes it easier for the user to identify appropriate straps for optimum functionality of the oral appliance.

In an alternative embodiment, the user is provided with a single strap having previously determined optimal dimensions.

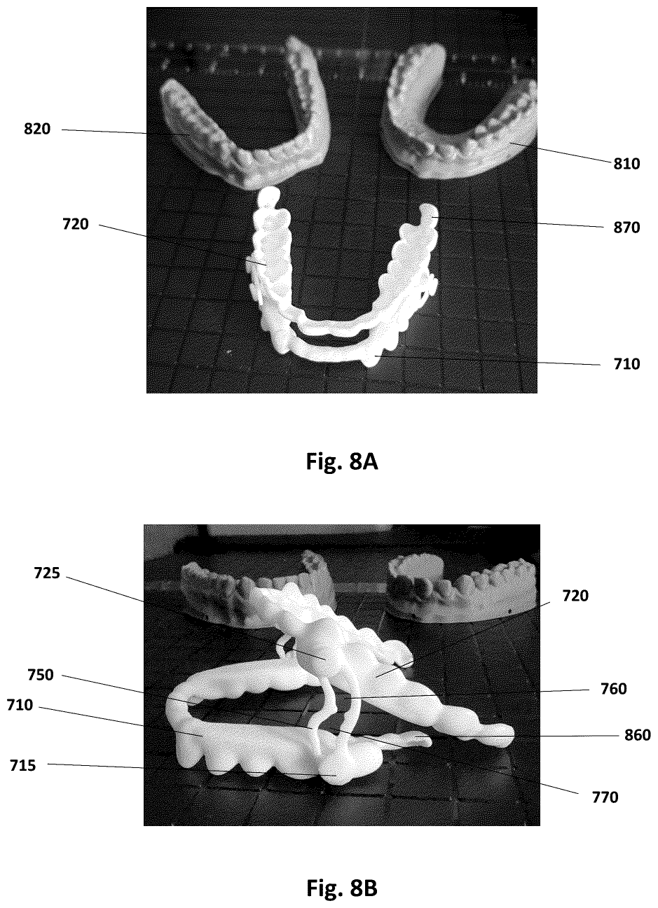

FIG. 8B illustrates an optional but preferred configuration of strap 750 having a slot recess connecting the pin holes to provide sliding engagement between retainer pins 715, 725 in slot 760, thus facilitating better flexibility of movement between maxillary and mandibular jaws of the user in forward-backward, upward-downward, and lateral (leftward-rightward) directions. The flexibility provided by the straps and the mandibular and maxillary parts in combination with the slots facilitate even angular positions relative to each other that may be in-between the aforementioned orthogonal directions. Moreover, for example, it may be possible to accommodate user specific requirements such as differential spacing between the maxillary and mandibular parts of the user on the left side and the right side.

Optionally, and in addition to the configurations described so far, a circular, elliptical, or a rectangular opening of increased size located at the middle region of the strap may be provided. In this embodiment, this larger opening 770 in the slot 760 of the connecting strap 750 is intended to allow a circular, elliptical or rectangular head of pin to be removed from or introduced to the strap, facilitating ease of change of strap. This configuration is incorporated to ameliorate the problem of unwanted disengagement between retainer pin and strap that may occur if the pin hole or slot connecting the pin holes is kept too large to facilitate the ease of change of strap. By providing a larger recess only at the middle region, the disengagement during use inside the oral cavity of the user is avoided. As the connecting strap is to be changed only outside the mouth of user, the maxillary and mandibular portions of the oral appliance can be freely moved to a relative position such that the pin and pin head can access the wider opening at the middle of a strap to facilitate removal of the strap from the pin. FIG. 8B shows opening 770 corresponding to an increase in slot width of the connector strap near the middle portion where pin may be advantageously moved to while the oral appliance is outside the oral cavity of a user to facilitate removal of the strap from the maxillary and mandibular portions and also facilitating engagement of alternative straps. Optionally, and in addition, the slot in the strap is configured to facilitate increased comfort for the user. This is achieved by providing a linear configuration of the slot near the maxillary part of the oral appliance that is approximately parallel to the upper jaw.

In one embodiment, the maxillary portion is configured to cover only a portion of the backmost molar of the upper dentition of the patient and the mandibular portion is configured to cover only a portion of the backmost molar of the lower dentition of the patient. In one embodiment, the portion of the backmost molar of the upper dentition covered by the oral appliance is the outer portion, and the portion of the backmost molar of the lower dentition is the inner portion. In an alternative embodiment, the portion of the backmost molar of the upper dentition is the inner portion, and the portion of the backmost molar of the lower dentition is the outer portion.

One embodiment of this feature is illustrated in FIGS. 8A and 8B. In FIG. 8A, where it can be seen that only the outer half 870 of the backmost portion of the oral appliance's maxillary portion 720 is provided. In FIG. 8B, it can be seen that only inner half 860 of the backmost portion of the oral appliance's mandibular portion is provided. Either configuration is considered advantageous to minimize crowding between the mandibular and maxillary portions of the oral appliance. These features individually or in combination ensure that the mandibular and maxillary jaws of the user are closer to each other when the user closes the jaws to contact each other while in sleep mode and is found to be more comfortable compared to jaws in relatively open condition due to interference between the backmost portions of the mandibular and maxillary teeth generally encountered.

In a further embodiment, the thickness of the backmost portions may be reduced relative to the forward portions of the oral appliance. This reduced thickness can also serve to reduce crowding or interference between two portions of the oral appliance.

In one embodiment, the components of the oral appliance, including maxillary and mandibular portions, pins and straps, are made of a light-weight flexible bio-compatible material. Those skilled in the art will appreciate that there are several bio-compatible materials that may be used to manufacture the components of the oral appliance, including but not limited to: Polyamides (e.g., Nylon-12, Nylon 6, Nylon 11, Nylon 66, Polyether Block Amide), Polyolefins (e.g., Polyethylene, Polypropylene, Cyclic Olefin Co-polymers, Polyvinyl Chloride), Polyesters (e.g., Poly butylene terephthalate, Poly ethylene terephthalate), Fluoropolymers (e.g., Poly tetrafluoroethylene known with trade name Teflon, PVDF, FEP, ePTFE), Elastomers (e.g., Silicones, Thermoplastic elastomers), Poly-p-xylylene (Parylene), Polystyrenes (e.g., Polyformaldehyde, Polyurethanes), Biopolymers, and several other combinations and reinforced hybrid materials under several manufacturer trade names. In a preferred embodiment, bio-compatible material is Nylon-12.

Furthermore, in addition, optionally the bio-compatible materials may be of several different colors, shades, and combinations of the same for those who prefer the same.

The oral appliance components, including the straps, may be manufactured by any suitable method as is known in the art, including but not limited to molding, additive manufacturing or 3D printing.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

We claim:

1. A system for diagnosing and treating obstructive sleep apnea (OSA) in a patient, the system comprising:
an oral appliance;
one or more biosensors configured to measure the one or more sleep parameters; and
a sleep application configured to monitor the one or more sleep parameters of the patient during sleep, wherein the application is configured to conduct one or more of a level 1, level 2, level 3 or level 4 sleep study;
wherein the system is configured to:
measure one or more sleep parameters while the patient is sleeping in a first sleep condition to obtain a first set of sleep parameters; and
measure one or more sleep parameters while the patient is sleeping in a second sleep condition to obtain a second set of sleep parameters;
compare the first and second sets of sleep parameters to quantify a change in sleep quality/patterns resulting from wearing the oral appliance; and
correlate the change in sleep quality/patterns with a diagnosis of OSA in the patient and a determination of the effectiveness of oral appliance therapy for treating the OSA.

2. The system of claim 1, wherein the first sleep condition comprises sleeping without the oral appliance and the second sleep condition comprises sleeping with the oral appliance.

3. The system of claim 1 or 2, wherein the oral appliance is a provisional oral appliance.

4. The system of claim 1, wherein the first sleep condition comprises sleeping with the oral appliance in a first state of adjustment, and the second sleep condition comprises sleeping with the oral appliance in a second state of adjustment.

5. The system of claim 4, wherein the first state of adjustment is a non-protrusive state where the upper jaw and lower jaw are in their natural relaxed relative positions, and the second state of adjustment is a protrusive state where the upper jaw and lower jaw are in positions displaced from their natural relative positions.

6. The system of claim 4 or 5, wherein the oral appliance is a provisional oral appliance or a custom oral appliance.

7. The system of claim 1, wherein the oral appliance is a heat moldable provisional oral appliance.

8. The system of claim 1, wherein the oral appliance comprises:
a lower mandibular portion adapted for fitting engagement with a lower dentition of the patient;
an upper maxillary portion adapted for fitting engagement with an upper dentition of the patient; and
a connection system configured to connect the upper maxillary portion and the lower mandibular portion to limit the range of movement of the maxillary portion relative to the mandibular portion, wherein the connection system comprises:
at least one connecting strap configured for attachment at a first end to the upper maxillary portion and at a second end to the lower mandibular portion,
wherein the at least one connecting strap is attached to the upper maxillary portion and the lower mandibular portion via retainer pins located on an outer side of the upper maxillary portion and on an outer side of the lower mandibular portion.

9. The system of claim 8, wherein the connecting straps are formed with slots to facilitate movement between the mandibular portion and the maxillary portion.

10. The system of claim 9, wherein the slots further comprise an enlarged opening in the middle to facilitate ease of removal of the connecting strap from the retainer pin.

11. The system of any one of claims 8 to 10, wherein each of said retainer pins is formed integrally as part of a respective mandibular or maxillary portion.

12. The system of any one of claims 8 to 11, wherein the maxillary portion is configured to cover only a portion of the backmost molar of the upper dentition of the patient and the mandibular portion is configured to cover only a portion of the backmost molar of the lower dentition of the patient.

13. The system of claim 12, wherein the portion of the backmost molar of the upper dentition is the outer portion, and the portion of the backmost molar of the lower dentition is the inner portion.

14. The system of claim 12, wherein the portion of the backmost molar of the upper dentition is the inner portion, and the portion of the backmost molar of the lower dentition is the outer portion.

15. The system of any one of claims 1 to 14, wherein the system is configured to measure one or more sleep parameters while the patient is sleeping in multiple different sleep conditions.

16. The system of any one of claims 1 to 15, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring.

17. The system of any one of claims 1 to 16, wherein the sleep application is a mobile application configured to interface with a smart device configured to measure the one or more sleep parameters using one or more biosensors.

18. The system of claim 17, wherein the smart device is a wearable smart device selected from a smart watch, a chest strap, a fingertip monitor, and/or the oral appliance.

19. The system of claim 17, wherein the smart device is a non-wearable smart device located in proximity to the sleeping patient.

20. The system of any one of claims 1 to 19, further comprising a kit for preparation of a mold of the patient's teeth.

21. The system of claim 20, wherein the mold is used to manufacture a custom oral appliance.

22. The system of claim 20, wherein the mold is scanned to provide a 3-D digital image which is used to manufacture a custom oral appliance, wherein the 3-D digital image is provided as an STL file or a 3D file format.

23. The system of claim 22, wherein the 3-D scan of the mold is obtained using a CT scanner or a cone beam CT (CBCT) scanner

24. The system of any one of claims 1 to 23, further comprising a tele-health platform configured to provide an interface with a health care professional, whereby the health care professional can monitor the one or more sleep parameters using the tele-health platform.

25. A process for diagnosing and treating obstructive sleep apnea (OSA) in a patient, comprising the steps of:
providing an oral appliance to be worn by the patient;
conducting a sleep study of the patient while sleeping, wherein the sleep study comprises:
measuring one or more sleep parameters while the patient is sleeping in a first sleep condition to obtain a first set of sleep parameters; and
measuring one or more sleep parameters while the patient is sleeping in a second sleep condition to obtain a second set of sleep parameters;
comparing the first and second sets of sleep parameters to quantify a change in sleep quality/patterns resulting from wearing the oral appliance; and
correlating the change in sleep quality/patterns with a diagnosis of OSA in the patient and a determination of the effectiveness of oral appliance therapy (OAT) for treating the OSA.

26. The process of claim 25, wherein the first sleep condition comprises sleeping without the oral appliance and the second sleep condition comprises sleeping with the oral appliance.

27. The process of claim 25 or 26, wherein the oral appliance is a provisional oral appliance.

28. The process of claim 25, wherein the first sleep condition comprises sleeping with the oral appliance in a first state of adjustment, and the second sleep condition comprises sleeping with the oral appliance in a second state of adjustment.

29. The process of claim 28, wherein the first state of adjustment is a non-protrusive state where the upper jaw and lower jaw are in their natural relaxed relative positions, and the second state of adjustment is a protrusive state where the upper jaw and lower jaw are in unnatural relative positions.

30. The process of claim 28 or 29, wherein the oral appliance is a provisional oral appliance or a custom oral appliance.

31. The process of claim 25, wherein the oral appliance comprises:
a lower mandibular portion adapted for fitting engagement with a lower dentition of the patient;
an upper maxillary portion adapted for fitting engagement with an upper dentition of the patient; and
a connection system configured to connect the upper maxillary portion and the lower mandibular portion to limit the range of movement of the maxillary portion relative to the mandibular portion, wherein the connection system comprises:
at least one connecting strap configured for attachment at a first end to the upper maxillary portion and at a second end to the lower mandibular portion,
wherein the at least one connecting strap is attached to the upper maxillary portion and the lower mandibular portion via retainer pins located on an outer side of the upper maxillary portion and on an outer side of the lower mandibular portion.

32. The process of claim 31, wherein the connecting straps are formed with slots to facilitate movement between the mandibular portion and the maxillary portion.

33. The process of claim 32, wherein the slots further comprise an enlarged opening in the middle to facilitate ease of removal of the connecting strap from the retainer pin.

34. The process of any one of claims 31 to 33, wherein each of said retainer pins is formed integrally as part of a respective mandibular or maxillary portion.

35. The process of any one of claims 31 to 34, wherein the maxillary portion is configured to cover only a portion of the backmost molar of the upper dentition of the patient and the mandibular portion is configured to cover only a portion of the backmost molar of the lower dentition of the patient.

36. The process of claim 35, wherein the portion of the backmost molar of the upper dentition is the outer portion, and the portion of the backmost molar of the lower dentition is the inner portion.

37. The process of claim 35, wherein the portion of the backmost molar of the upper dentition is the inner portion, and the portion of the backmost molar of the lower dentition is the outer portion.

38. The process of any one of claims 25 to 37, wherein the change in sleep quality/patterns indicates an improvement in sleep quality, indicating a diagnosis of OSA that is treatable using an oral appliance.

39. The process of claim 38, wherein the oral appliance is a custom oral appliance.

40. The process of any one of claims 25 to 39, further comprising the steps of measuring one or more sleep parameters while the patient is sleeping in multiple different sleep conditions.

41. The process of any one of claims 25 to 40, wherein the determination of the effectiveness of OAT for treating the OSA includes a determination of the effectiveness of a provisional oral appliance for treatment of the OSA with OAT.

42. The process of any one of claims 25 to 40, wherein the determination of the effectiveness of OAT for treating the OSA includes a prediction of the effectiveness of a custom oral appliance for treatment of the OSA with OAT.

43. The process of any one of claims 25 to 42, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring.

44. The process of any one of claims 25 to 43, wherein the one or more sleep parameters are measured using one or more biosensors, provided in a wearable smart device selected from a smart watch, a chest strap, a head strap, a fingertip monitor, and/or in the oral appliance.

45. The process of any one of claims 25 to 44, wherein upon a positive diagnosis of OSA in the patient, the process further comprises:
providing an impression kit to make a mold of the patient's teeth, and
using the mold to manufacture a custom oral appliance.

46. The process of any one of claims 25 to 44, wherein upon a positive diagnosis of OSA in the patient and a determination of the effectiveness of OAT, the process further comprises:
obtaining a 3-D digital scan of the patient's teeth, and
using the 3-D digital scan to manufacture a custom oral appliance.

47. The process of claim 45 or 46, wherein the custom oral appliance comprises one or more biosensors, wherein the custom oral appliance is configured to measure one or more sleep parameters while the patient is sleeping with the custom oral appliance.

48. The process of any one of claims 25 to 47, further comprising the step of providing a tele-health platform configured to provide an interface with a health care professional, whereby the health care professional can monitor the one or more sleep parameters using the tele-health platform.

49. A custom oral appliance for use in diagnosing sleep apnea in a subject wearing the appliance, wherein the oral appliance comprises:
a lower mandibular portion adapted for fitting engagement with a lower dentition of the patient;
an upper maxillary portion adapted for fitting engagement with an upper dentition of the patient; and
a connection system configured to connect the upper maxillary portion and the lower mandibular portion to limit the range of movement of the maxillary portion relative to the mandibular portion, wherein the connection system comprises:
at least one connecting strap configured for attachment at a first end to the upper maxillary portion and at a second end to the lower mandibular portion,
wherein the at least one connecting strap is attached to the upper maxillary portion and the lower mandibular portion via retainer pins located on an outer side of the upper maxillary portion and on an outer side of the lower mandibular portion;
wherein the oral appliance is configured for use in conjunction with one or more biosensors configured to measure one or more sleep parameters, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring.

50. The oral appliance of claim 49, wherein the connecting straps are formed with slots to facilitate movement between the mandibular portion and the maxillary portion.

51. The oral appliance of claim 50, wherein the slots further comprise an enlarged opening in the middle to facilitate ease of removal of the connecting strap from the retainer pin.

52. The oral appliance of any one of claims 49 to 51, wherein each of said retainer pins is formed integrally as part of a respective mandibular or maxillary portion.

53. The oral appliance of any one of claims 49 to 52, wherein the maxillary portion is configured to cover only a portion of the backmost molar of the upper dentition of the patient and the mandibular portion is configured to cover only a portion of the backmost molar of the lower dentition of the patient.

54. The oral appliance of claim 53, wherein the portion of the backmost molar of the upper dentition is the outer portion, and the portion of the backmost molar of the lower dentition is the inner portion.

55. The oral appliance of claim 53, wherein the portion of the backmost molar of the upper dentition is the inner portion, and the portion of the backmost molar of the lower dentition is the outer portion.

56. The oral appliance of any one of claims 49 to 55, wherein at least one of the one or more biosensors is incorporated into the oral appliance.

57. A kit for diagnosing obstructive sleep apnea (OSA) in a patient, the kit comprising:
equipment for preparing an oral appliance as defined in claim 40;
one or more biosensors configured to measure one or more sleep parameters;
a sleep application configured to monitor the one or more sleep parameters of the patient during sleep, wherein the application is configured to conduct one or more of a level 1, level 2, level 3 or level 4 sleep study; and
instructions for use.

58. The kit of claim 57, wherein the one or more sleep parameters are selected from heart rate, breathing rate, head movement, body movement, bite force, body temperature, actigraphy, oximetry, apnea hypopnea index (AHI), peripheral arterial tone (PAT), cardiac data, chest expansion, respiratory air flow, sleeping position, and snoring.

59. The kit of claim 57 or 58, wherein the sleep application is a mobile application configured to interface with a wearable smart device configured to measure the one or more sleep parameters using one or more biosensors.

60. The kit of claim 59, wherein the wearable smart device is selected from a smart watch, a chest strap, head strap, a fingertip monitor, and/or the provisional oral appliance.

61. The kit of any one of claims 57 to 60, wherein the equipment for preparing the oral appliance comprises an impression kit for preparation of a mold of the patient's teeth.

62. The kit of claim 61, wherein the mold is used to manufacture a custom oral appliance.

63. The kit of claim 61, wherein the mold is scanned to provide a 3-D digital image which is used to manufacture a custom oral appliance.

64. The kit of claim 63, further comprising one or more of a sleep mask, ear plugs, cleaning kit, a bite repositioner, and chewing gum.

65. The kit of any one of claims 57 to 64, wherein the equipment for preparing the oral appliance comprises an imaging device for obtaining a 3-D intra-oral digital image, wherein the 3-D digital image is provided as an STL file or as a 3D file format.

66. The kit of claim 65, wherein the imaging device is configured for communication with a device such as a smart phone, tablet or personal computer, and wherein the scanning process is managed using a mobile application on the device.

67. The kit of claim 66, wherein the communication between the imaging device and the device is carried out on a wireless connection, a Bluetooth connection, or a wired connection.
